(19) United States
(12) Patent Application Publication
(10) Pub. No.: US 2008/0221461 A1
(43) Pub. Date: Sep. 11, 2008

(54) VITAL SIGN MONITOR FOR CUFFLESSLY MEASURING BLOOD PRESSURE WITHOUT USING AN EXTERNAL CALIBRATION

(75) Inventors: Zhou Zhou, La Jolla, CA (US); Marshal Singh Dhillon, San Diego, CA (US); Henk Visser, San Diego, CA (US); Matthew John Banet, Del Mar, CA (US); Andrew Stanley Terry, San Diego, CA (US)

Correspondence Address: Triage Wireless, Inc.;Matthew John Banet, 9444 Waples Street, Suite 280, SAN DIEGO, CA, 92121, US

(73) Assignee: TRIAGE WIRELESS, INC., San Diego, CA (US)

(21) Appl. No.: 11/682,228

(22) Filed: Mar. 5, 2007

(51) Int. Cl. A61B 5/021

(52) U.S. Cl. 600/485

(57) ABSTRACT

The invention provides a method for measuring a patient's blood pressure featuring the following steps: 1) measuring a first time-dependent optical signal with a first optical sensor; 2) measuring a second time-dependent optical signal with a second optical sensor; 3) measuring a time-dependent electrical signal with an electrical sensor; 4) estimating the patient's arterial properties using either the first or second time-dependent optical signal; 5) determining a pulse transit time (PTT) from the time-dependent electrical signal and at least one of the first and second time-dependent optical signals; and 6) calculating a blood pressure value using a mathematical model that includes the PTT and the patient's arterial properties.

### CROSS REFERENCES TO RELATED APPLICATION

Not Applicable

### STATEMENT REGARDING FEDERALLY SPONSORED RESEARCH OR DEVELOPMENT

Not Applicable

### BACKGROUND OF THE INVENTION

1. Field of the Invention

The present invention relates to medical devices for monitoring vital signs, e.g., arterial blood pressure.

2. Description of the Related Art

Pulse transit time (‘PTT’), defined as the transit time for a pressure pulse launched by a heartbeat in a patient's arterial system, has been shown in a number of studies to correlate to both systolic and diastolic blood pressure. In these studies, PTT is typically measured with a conventional vital signs monitor that includes separate modules to determine both an electrocardiogram (‘ECG’) and pulse oximetry. During a PTT measurement, multiple electrodes typically attach to a patient's chest to determine a time-dependent ECG characterized by a sharp spike called the ‘QRS complex’. This feature indicates an initial depolarization of ventricles within the heart and, informally, marks the beginning of the heartbeat and a pressure pulse that follows. Pulse oximetry is typically measured with a bandage or clothespin-shaped sensor that attaches to a patient's finger, or wrist, and includes optical systems operating in both the red and infrared spectral regions. A photodetector measures radiation emitted from the optical systems and transmitted through the patient's finger. Other body sites, e.g., the ear, forehead, and nose, can also be used in place of the finger or wrist. During a measurement a microprocessor analyses red and infrared radiation measured by the photodetector to determine the patient's blood oxygen saturation level and a time-dependent waveform called a plethysmograph. Time-dependent features of the plethysmograph indicate both pulse rate and a volumetric change in an underlying artery (e.g., in the finger) caused by the propagating pressure pulse.

Typical PTT measurements determine the time separating a maximum point on the QRS complex (indicating, i.e., the peak of ventricular depolarization) and a foot of the plethysmograph (indicating, i.e., initiation of the pressure pulse). PTT depends primarily on arterial compliance, the propagation distance of the pressure pulse (closely approximated by the patient's arm length), and blood pressure. For a given patient, PTT typically decreases with an increase in blood pressure and a decrease in arterial compliance. Arterial compliance, in turn, typically decreases with age.

A number of issued U.S. Patents describe the relationship between PTT and blood pressure. For example, U.S. Pat. Nos. 5,316,008; 5,857,975; 5,865,755; and 5,649,543 each describe an apparatus that includes conventional sensors that measure an ECG and plethysmograph, which are then processed to determine PTT.

Studies have also shown that a property called vascular transit time (‘VTT’), defined as the time separating two plethysmographs measured from different locations on a patient, can correlate to blood pressure. Alternatively, VTT can be determined from the time separating other time-dependent signals measured from a patient, such as those measured with acoustic or pressure sensors. A study that investigates the correlation between VTT and blood pressure is described, for example, in ‘Evaluation of blood pressure changes using vascular transit time’, Physiol. Meas. 27, 685-694 (2006). U.S. Pat. Nos. 6,511,436; 6,599,251; and 6,723,054 each describe an apparatus that includes a pair of optical or pressure sensors, each sensitive to a propagating pressure pulse, that measure VTT. As described in these patents, a microprocessor associated with the apparatus processes the VTT value to estimate blood pressure.

In order to accurately measure blood pressure, both PTT and VTT measurements typically require a ‘calibration’ consisting of one and more conventional blood pressure measurements made simultaneously with the PTT or VTT measurement. The calibration accounts for patient-to-patient variation in arterial properties (e.g., stiffness and size). Calibration measurements are typically made with an auscultatory technique (e.g., using a pneumatic cuff and stethoscope) at the beginning of the PTT or VTT measurement; these measurements can be repeated if and when the patient undergoes any change that may affect their physiological state.

Other efforts have attempted to use a calibration along with other properties of the plethysmograph to measure blood pressure. For example, U.S. Pat. No. 6,616,613 describes a technique wherein a second derivative is taken from a plethysmograph measured from the patient's ear or finger. Properties from the second derivative are then extracted and used with calibration information to estimate the patient's blood pressure. In a related study, described in ‘Assessment of Vasoactive Agents and Vascular Aging by the Second Derivative of Photoplethysmogram Waveform’, Hypertension. 32, 365-370 (1998), the second derivative of the plethysmograph is analyzed to estimate the patient's ‘vascular age’ which is related to the patient's biological age and vascular properties.

### SUMMARY OF THE INVENTION

This invention provides a medical device that makes a cuffless, non-calibrated measurement of blood pressure using PTT and a correction that accounts for the patient's arterial properties (e.g., stiffness and size). This correction, referred to herein as a ‘vascular index’ (‘VI’), is calculated according to one of two methods. In the first method, the VI is determined by analyzing the shape of the plethysmograph, measured at either the brachial, finger artery, or wrist. In this method, in order to accurately extract features from the shape of the plethysmograph, this waveform is typically first passed through a mathematical filter based on Fourier Transform (called the ‘Windowed-Sinc Digital Filter’) and then analyzed by taking its second derivative. In the second method, the VI is estimated from the VTT measured between the patient's brachial and finger arteries. In both cases, the VI is used in combination with the patient's biological age to estimate their arterial properties. These properties are then used to ‘correct’ PTT and thus calculate blood pressure without the need for an external calibration (e.g., without input of an auscultatory measurement).

This invention is based on the discovery that a PTT value corrected for the patient's arterial properties using age and VI shows a high correlation to blood pressure. Moreover, the correlation between PTT and blood pressure is further improved by measuring PTT using ECG and a plethysmograph measured from the patient's brachial artery (i.e., near the patient's elbow, anterior of the medial epicondyle). Due to the thickness of tissue in this region, the plethysmograph is best measured using a reflective optical sensor. In this configuration, the signal-to-noise ratio of the plethysmograph can be increased by using a multi-sensor array instead of a single sensor, and by choosing an optical wavelength (λ˜570 nm) that works well in a reflection-mode geometry for a variety of skin types.

PTT, VI and blood pressure, along with other information such as heart rate, heart rate variability, respiratory rate, pulse oximetry, pulse wave velocity (‘PWV’), and temperature, are analyzed with a hand-held device that includes many features of a conventional personal digital assistant (‘PDA’). The device includes, for example, a microprocessor that runs an icon-driven graphical user interface (‘GUI’) on a color, liquid crystal display (‘LCD’) attached to a touch panel. A user selects different measurement modes, such as continuous, one-time, and 24-hour ambulatory modes, by tapping a stylus on an icon within the GUI. The device also includes several other hardware features commonly found in PDAs, such as short-range (e.g., Bluetooth® and WiFi®) and long-range (e.g., CDMA, GSM, IDEN) modems, global positioning system (‘GPS’), digital camera, and barcode scanner.

In one aspect, for example, the invention provides a method for measuring a patient's blood pressure that includes the following steps: 1) measuring a first time-dependent optical signal with a first optical sensor; 2) measuring a second time-dependent optical signal with a second optical sensor; 3) measuring a time-dependent electrical signal from the heart with an electrical sensor; 4) determining a VI from either (or both) the first and second time-dependent optical signals; 4) determining a PTT from the time-dependent electrical signal from the heart and at least one of the first and second time-dependent optical signals; 5) correcting the PTT with the VI and the patient's biological age; and 6) calculating a blood pressure value using a mathematical model that includes the corrected PTT.

In embodiments, the method includes the step of determining the VI from either VTT or by analyzing the properties (taken, e.g., from the second derivative) of either the first or second optical signals. To measure the optical signals, for example, the first optical sensor can operate in a transmission or reflection-mode geometry on the patient's finger near the digital artery, and the second optical sensor can operate can operate in a reflection-mode geometry within a sensor armband positioned near the patient's brachial or radial artery. In other embodiments, the electrical sensor features at least two electrodes (for ECG data), with one electrode typically attached to the patient's chest, and the second electrode typically embedded within the sensor armband.

In other embodiments, the method includes the step of estimating the patient's arterial properties by comparing the VTT (or a mathematic equivalent thereof, such as PWV) to a predetermined look-up table or mathematical function. Both the look-up table and mathematical function relate the VTT or PWV to an arterial property, or alternatively to a ‘figure of merit’ representing a collective arterial property, e.g., a combination of properties representative of the patient's arterial vasculature.

In other embodiments, the method includes determining PTT by analyzing a first time-dependent feature from the time-dependent electrical signal from the heart and a second time-dependent feature from either the first or the second time-dependent optical signal. For example, the first time-dependent feature can be a peak of a QRS complex within the time-dependent electrical signal from the heart, and the second time-dependent feature can be base of an optical plethysmograph.

The invention has a number of advantages. In general, the device described herein uses both PTT and VI to make a cuffless measurement of blood pressure without requiring calibration at the beginning of the measurement. This dramatically simplifies the process of measuring blood pressure without using a cuff. Moreover, the device combines all the data-analysis features and form factor of a conventional PDA with the monitoring capabilities of a conventional vital sign monitor. This results in an easy-to-use, flexible device that performs one-time, continuous, and ambulatory measurements both in and outside of a hospital. And because it lacks a pneumatic cuff or any type of calibration, the device measures blood pressure in a simple, rapid, and pain-free manner. Measurements can be made throughout the day with little or no inconvenience to the caregiver or patient. Moreover, the optical and electrical sensors can be integrated into or connected to a comfortable armband that wirelessly communicates with device. This eliminates the wires that normally tether a patient to a conventional vital sign monitor, thereby increasing patient comfort and enabling mobility.

These and other advantages are described in detail in the following description, and in the claims.

### BRIEF DESCRIPTION OF THE DRAWINGS

FIG. 1 shows a schematic drawing of the device and sensor armband of the invention attached to a patient;

FIG. 2 shows a schematic side view of the armband of FIG. 1 attached to the arm of the patient;

FIG. 3A shows a schematic cross-sectional view of the armband of FIG. 2 attached to the arm of the patient;

FIG. 3B shows a schematic side view of the armband of FIGS. 1, 2 and 3A;

FIG. 4 shows a mathematical equation describing how blood pressure can be calculated from PTT and a VI measured using one of two methods;

FIGS. 5A, 5B, and 5C show graphs of, respectively, a plethysmograph measured from a patient (FIG. 5A); the plethysmograph of FIG. 5A filtered with a Windowed-Sinc Digital Filter (FIG. 5B); and the second derivative of the digitally filtered plethysmograph shown in FIG. 5B (FIG. 5C);

FIG. 6 shows a graph of a second derivative of a digitally filtered plethysmograph (black line) and an unfiltered plethysmograph (gray line);

FIG. 7 shows a graph of a second derivative of a digitally filtered plethysmograph including time-dependent features used in a VI calculation;

FIGS. 8A and 8B show, respectively, optical and electrical waveforms processed according to the invention to measure PTT, and two optical waveforms processed according to the invention to measure VTT;

FIGS. 9A, 9B, and 9C are graphs determined from a 110-patient study showing correlation between systolic blood pressure measured with a auscultatory method and, respectively, PTT2, PTT1, and VTT;

FIG. 10 is a schematic drawing of a human body showing arterial path lengths corresponding to the values of PTT2, PTT1, and VTT used in, respectively, FIGS. 9A, 9B, and 9C;

FIGS. 11A and 11B are graphs determined from a 4-patient study showing, respectively, the correlation between diastolic and mean blood pressure, and the correlation between diastolic and systolic blood pressure;

FIG. 12 is a flow chart showing an algorithm used to measure blood pressure by analyzing PTT and VI; and,

FIG. 13 is a schematic view of a patient wearing a sensor armband of FIG. 1 communicating with the device of FIG. 1, which is mounted in a docking station.

### DETAILED DESCRIPTION OF THE INVENTION

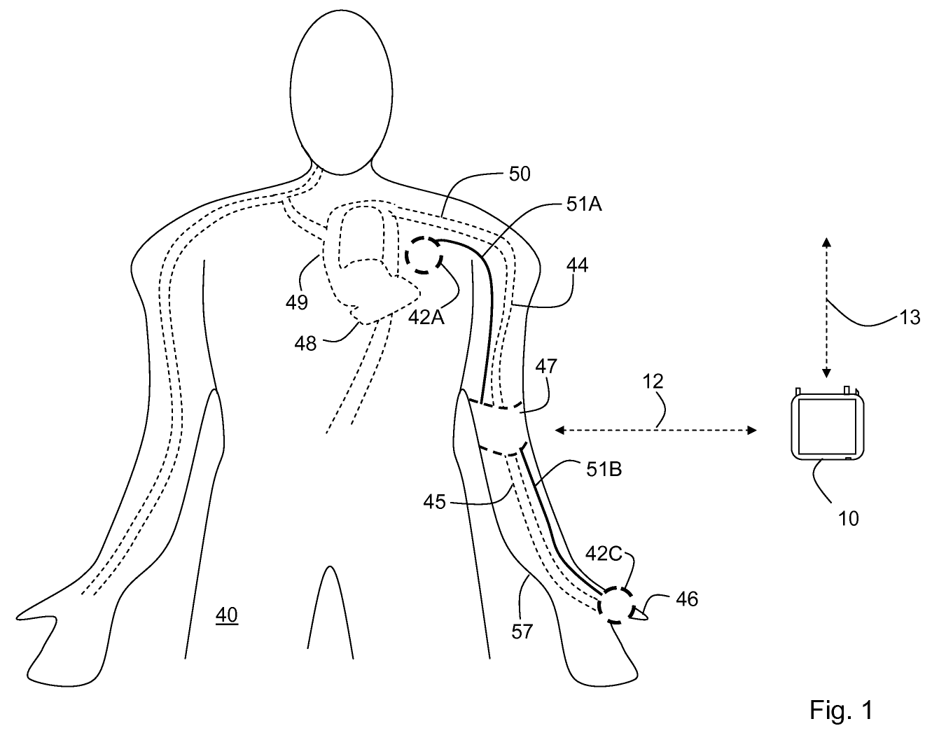

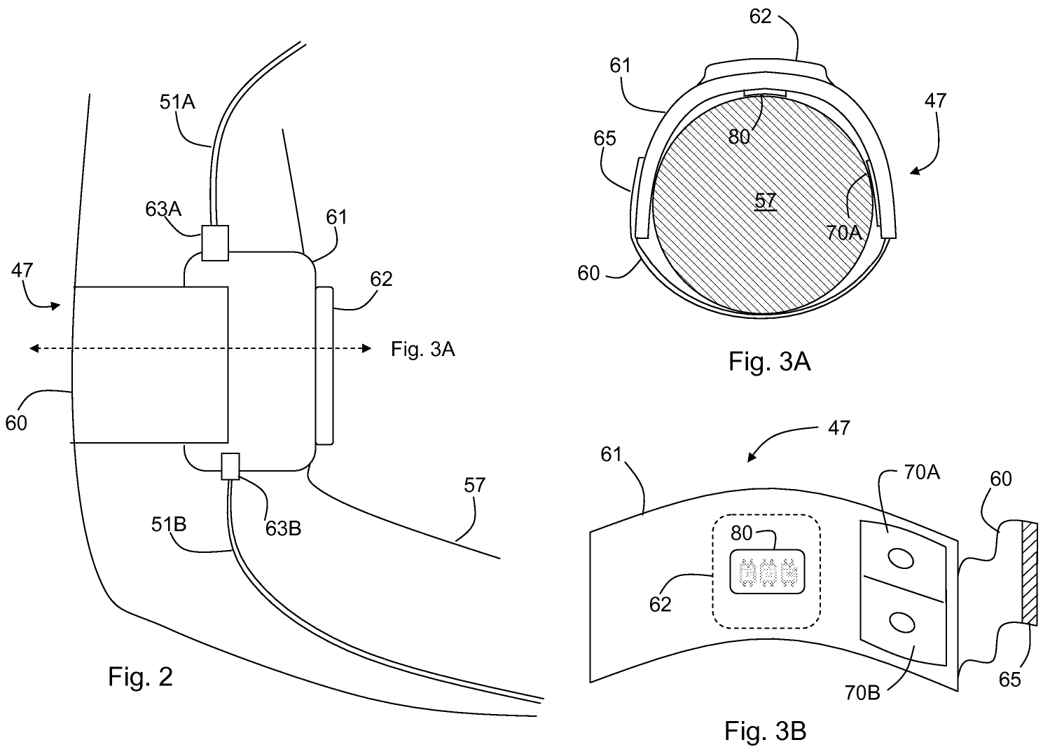

FIGS. 1, 2, 3A, and 3B show a system that measures blood pressure from a patient 40 using PTT, VI, and no external calibration. Prior to the measurement, a medical professional or patient places a sensor armband 47 on the patient's arm 57 near their elbow. The armband 47 includes an optical sensor array 80 and a 2-part adhesive electrode 70A, 70B that both attach to a thick foam band 61. A flexible strap 60 featuring a Velcro® portion 65 secures the sensor armband 47 to the patient's arm 57 so that the array of optical sensors 80 and the 2-part adhesive electrode 70A, 70B contact the patient's skin. Preferably the 2-part adhesive electrode 70A, 70B is a disposable component that snaps into a matched receptacle in the sensor armband 47 and includes an adhesive backing, while the array of optical sensors 80 is a non-disposable component featuring multiple optical modules. The optical sensor array 80 is preferably disposed above the patient's brachial artery 44, while the electrodes 70A, 70B are less sensitive to position, and simply need to maintain skin contact. A secondary electrode 42A attaches to the patient's chest and connects to the armband 47 through a first cable 51A. A secondary sensor 42C featuring a pulse oximeter and additional optical sensor connects to the armband 47 through a second cable 51B. In a preferred embodiment, the secondary electrode 42A and 2-part adhesive electrodes are custom-made ECG electrodes, and the optical sensors within the optical sensor array 80 are integrated modules, each featuring a light source and a photodetector. These sensors are described in detail below. Both the first 51A and second 51B cables connect to an electronics module 62 embedded in the sensor armband 47 through a pair of stereo-jack connectors 63A, 63B that allows these cables 51A, 51B to be easily detached.

The patient's heart 48 generates electrical impulses that pass through the body near the speed of light. These impulses stimulate each heart beat, which in turn generates a pressure wave that propagates through the patient's vasculature at a significantly slower speed. Immediately after the heartbeat, the pressure wave leaves the aorta 49, passes through the subclavian artery 50, to the brachial artery 44, and from there through the radial artery 45 to smaller arteries in the patient's fingers. During a measurement, the two-part electrode 70A, 70B in the sensor armband 47 and in the secondary sensor 42A measure unique electrical signals which pass to an amplifier/filter circuit included in the embedded electronics module 62. There, the signals are processed using the amplifier/filter circuit to determine an analog ECG signal, which is then digitized with an analog-to-digital converter and stored in memory in a microprocessor. Using reflection-mode geometry, the optical sensor array 80 in the sensor armband 47 and the optical module in the secondary sensor 42C measure, respectively, analog plethysmographs from the patient's brachial and finger arteries. These signals are amplified using second and third amplifier/filter circuits and digitized with second and third channels within the analog-to-digital converter in the electronics module 62. Each plethysmograph features a time-dependent ‘pulse’ corresponding to each heartbeat that represents a volumetric change in an underlying artery caused by the propagating pressure pulse.

The optical modules within the optical sensor array 80 typically include an LED operating near 570 nm, a photodetector, and an amplifier. This wavelength is selected because it is particularly sensitive to volumetric changes in an underlying artery when deployed in a reflection-mode geometry, as described in the following co-pending patent application, the entire contents of which are incorporated herein by reference: SYSTEM FOR MEASURING VITAL SIGNS USING AN OPTICAL MODULE FEATURING A GREEN LIGHT SOURCE (U.S. Ser. No. 11/307,375; filed Feb. 3, 2006). A preferred optical module is the TRS 1755 manufactured by TAOS Inc. of Plano, Tex. (www.taosinc.com). Typically, three optical modules are used in the sensor array 80 to increase the probability that an underlying artery is measured, thus increasing the signal-to-noise ratio of the measurement. Operating in concert, the three sensors collectively measure an optical signal that includes photocurrent generated by each optical module. The resultant signal effectively represents an ‘average’ signal measured from vasculature (e.g., arteries and capillaries) underneath the sensor array 80. The secondary sensor 42C includes a similar optical module, and additionally includes LEDs operating near 650 nm and 950 nm in order to make a pulse oximetry measurement.

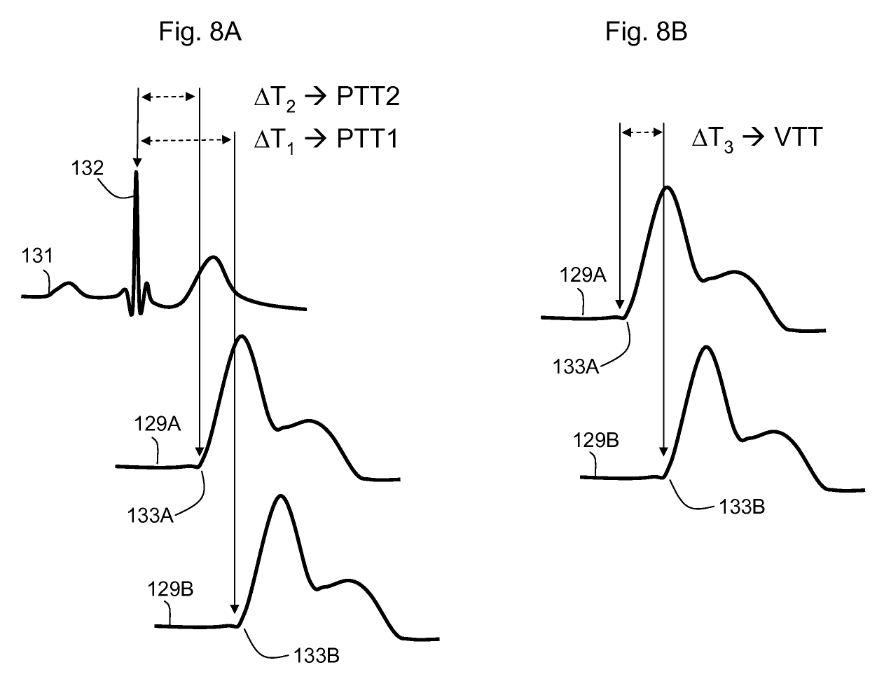

FIGS. 8A and 8B show, for example, the digitized ECG signal 131, plethysmograph measured from the brachial artery 129A, and plethysmograph measured from the finger artery 129B as described above. In a preferred embodiment, software running on the microprocessor within the device simultaneously determines three transit times from these waveforms. The first pulse transit time (‘PTT1’) is determined from the time separating a spiked, QRS complex 132 within the ECG signal 131 and a foot 133B of the plethysmograph measured from the finger artery 129B. The second pulse transit time (‘PTT2’) is determined from the time separating the QRS complex 132 and a foot 133A of the plethysmograph measured from the brachial artery 129A. Finally, the vascular transit time (‘VTT’) is determined from the time separating the foot 133A of the plethysmograph from the brachial artery 129A and the foot 133B of the plethysmograph from the finger artery 129B. Pulse wave velocity (‘PWV’) is determined by dividing the distance separating the sensors used to measure the two plethysmographs by VTT.

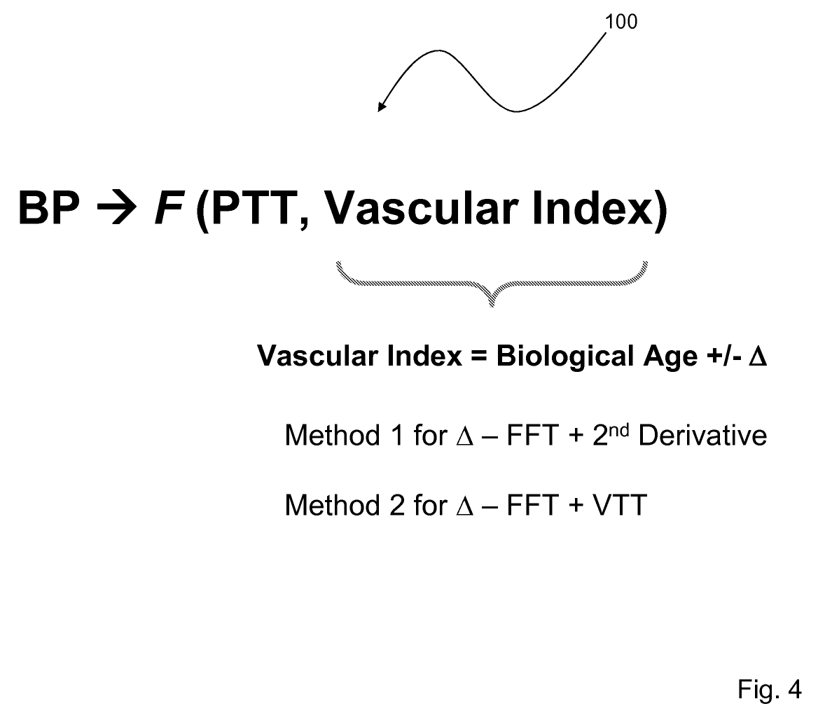

The device determines the patient's blood pressure using the transit times shown schematically in FIGS. 8A and 8B and VI. FIG. 4 shows a mathematical equation 100 that indicates how blood pressure is calculated from PTT and VI, which can be estimated using two separate methods. In both methods, VI is equivalent to the patient's biological age adjusted by a ‘Δ’ factor determined by filtering one of the above-described plethysmographs by a Windowed-Sinc Digital Filter using a fast Fourier Transform (‘FFT’) and then analyzed using either a second derivative (Method 1), or by using VTT (Method 2). Typically, it is the plethysmograph measured from the patient's finger (129B in FIGS. 8A and 8B) used in this analysis, as this signal originates mostly from the finger artery, while the signal measured from the brachial artery may have contributions from capillaries located just below the patient's skin.

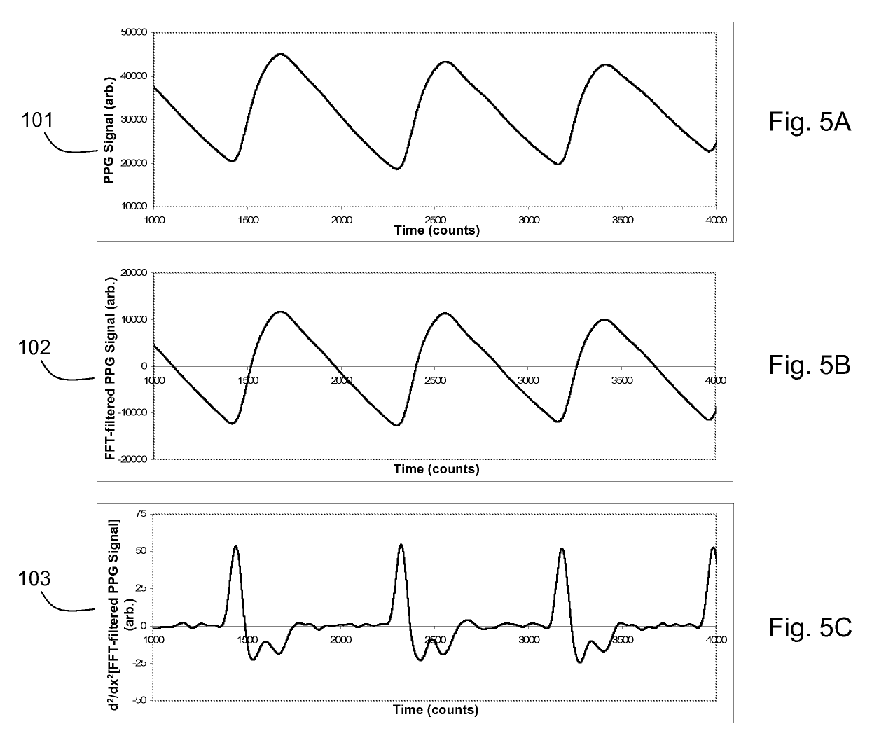

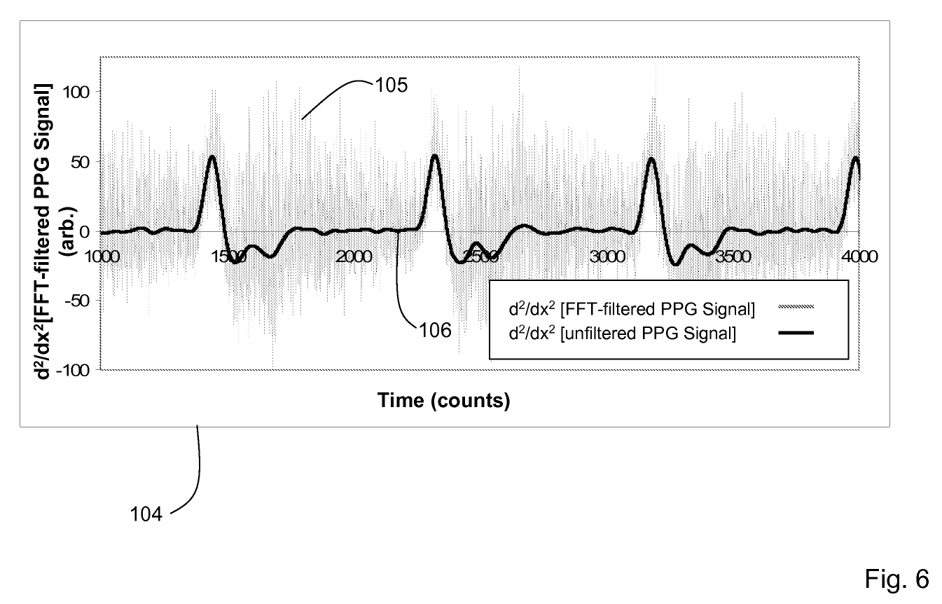

Using Method 1, the patient's VI is estimated from features contained within the second derivative of the plethysmograph measured from the patient's finger. As shown in FIGS. 5A, 5B, and 5C, for this analysis the plethysmograph is first measured and digitized using the analog-to-digital converter within the electronics module in the sensor armband. FIG. 5A, for example, shows the digitized plethysmograph. To remove extraneous noise, the plethysmograph is filtered using an FFT-based algorithm as described above; this algorithm typically passes frequencies between 0.1 and 15 Hz, and rejects filters any frequencies outside of this range (from e.g., non-physiologic sources). FFT-based digital filtering algorithms are well known in signal processing, and are described for example in: Numerical Recipes in C, 1988, Cambridge University Press, the contents of which are incorporated by reference. FIG. 5B, for example, shows a graph 101 of the resultant filtered plethysmograph resulting from this filtering process. The baseline of the waveform in FIG. 5A is composed primarily of low-frequency components which are filtered as described above; this is why the baseline of the waveform in the graph 102 of FIG. 5B is centered on zero. Once these frequencies are removed, the resulting plethysmograph is derivatized twice to generate a second derivative, shown in the graph 103 of FIG. 5C, that includes time-dependent features sensitive to the stiffness of the patient's arteries. If the original waveform is not digitally filtered, small amounts of noise in the unfiltered plethysmograph are amplified once the waveform is derivatized. FIG. 6, for example, shows the second derivative of both digitally filtered (black trace 106) and unfiltered (gray trace 105) waveforms. As is clear from this figure, unfiltered noise is amplified after taking the second derivative, making it difficult to analyze the resulting waveform for the desired signal. In contrast, the derivatized digitally filtered waveform has an extremely high signal-to-noise ratio, making it significantly easier to analyze for the desired signal. Ultimately, this results in a relatively accurate measurement of VI and, ultimately, blood pressure.

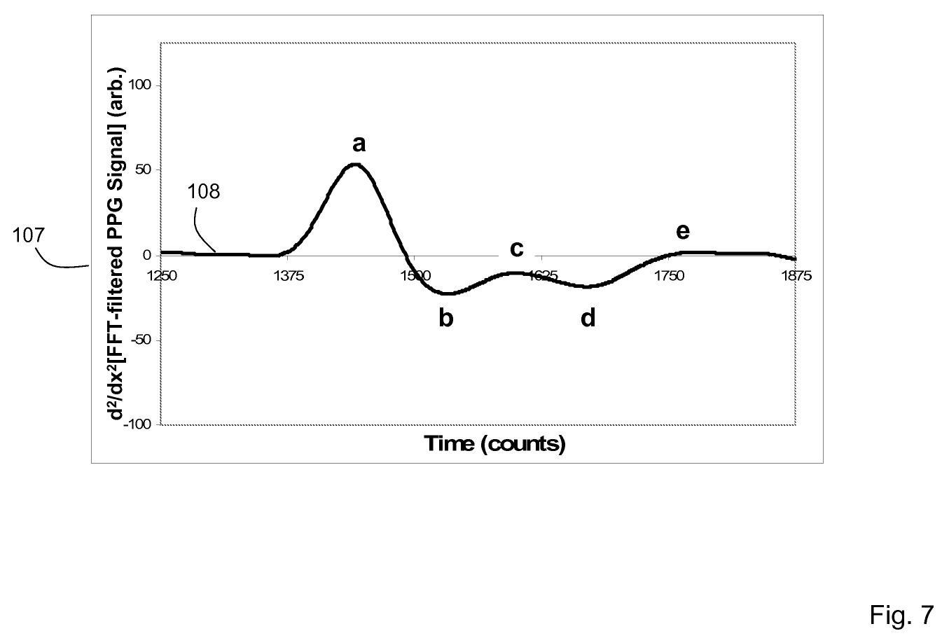

FIG. 7 shows in more detail the features of the second derivative of the finger plethysmograph, labeled ‘a’, ‘b’, ‘c’, ‘d’, and ‘e’, used to calculate VI. They can be related to VI using equation 1, below:

VI=biological age+A₁*[(b−c−d−e)/a] 1)

where A₁ is a predetermined constant and biological age is the patient's actual age in units of years. PTT can then be corrected using VI according to equation 2, below:

PTT (corrected)=PTT (uncorrected)+VI 2)

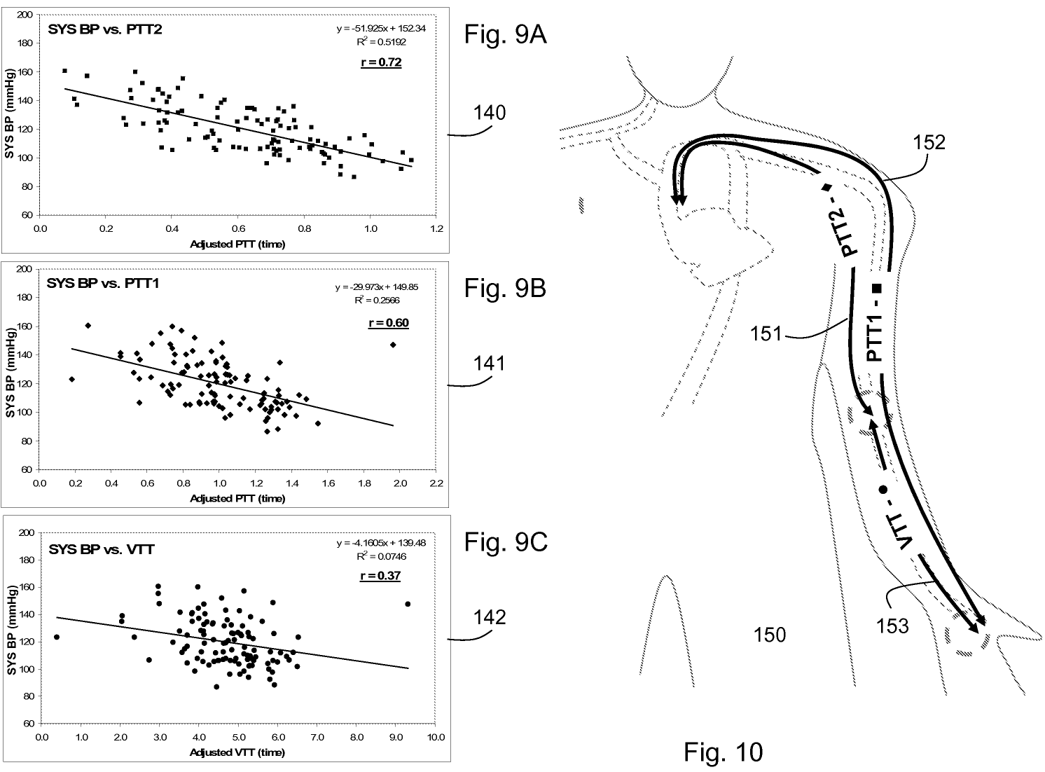

With this correction, PTT can be measured and used to calculate blood pressure without requiring any external calibration, as described in more detail with reference to FIGS. 9A, 9B, 9C, and 10.

Method 2 is alternative way to calculate VI using VTT, and is based on the assumptions that, compared to PTT, VTT and PWV are relatively sensitive to a patient's arterial properties. This assumption is based on a statistical comparison between cuff-based blood pressure, VTT, and PTT values generated from a 110-patient study, described in more detail below. VTT can therefore be used to estimate VI, as shown in Equation 3 below.

VI=biological age+A₂*VTT 3)

where A₂ is a predetermined constant and biological age is as described above. PTT can then be corrected using VI according to equation 2, above.

Once corrected with VI, PTT can be used to calculate systolic and mean arterial blood pressure (without requiring any external calibration) using a simple linear equation, as described in Equations 4 and 5 below:

systolic blood pressure=M_{SYS}*PTT(corrected)+B_{SYS} 4)

mean arterial blood pressure=M_{MAP}*PTT(corrected)+B_{MAP} 5)

Where M_{SYS}, M_{MAP}, B_{SYS}, and B_{MAP} are constants of linear equations determined empirically from a large study population. Diastolic blood pressure is determined from mean arterial blood pressure as described in more detail below.

FIGS. 9A, 9B, and 9C show graphs 140, 141, 142 taken from a 110-patient study wherein transit times simultaneously measured with the sensor armband (47 in FIG. 1) of the invention are compared to systolic blood pressure. A high correlation between the transit time and blood pressure in this type of study indicates that the transit time can determine blood pressure without the need for an external calibration (e.g., a pre-measurement auscultatory technique). As shown in FIG. 10 and described above, during the study, the sensor armband simultaneously measures two pulse transit times (PTT1, PTT2) and one vascular transit time (VTT) from each of the 110 patients. As shown in FIG. 9A, PTT2 is measured between the onset of the QRS complex in the ECG and the foot of a plethysmograph measured near the brachial artery. PTT2 correlates better with systolic blood pressure (r=0.72) compared with PTT1 (r=0.60), which is measured using a finger plethysmograph and is shown in FIG. 9B. Both PTT1 and PTT2 correlate better with systolic blood pressure than VTT (r=0.37), which is measured using plethysmographs from the brachial and finger arteries, as shown in FIG. 9C. Without being bound to any theory, this improved correlation may be due to the fact that PTT2 corresponds to a transit time for a pressure pulse propagating along a pathway 151 through relatively larger arteries (i.e., the aortic, subclavian, and brachial arteries) that have relatively small surface-to-volume ratios. A pressure pulse propagating through this large pathway 151 may be less affected by the arteries' mechanical properties (e.g., stiffness, size) than a pulse propagating along a pathway 152 that includes smaller arteries (i.e., the radial and finger arteries) that have a relatively small surface-to-volume ratio. The pathway 153 corresponding to the VTT measurement is composed entirely of the relatively small radial and finger arteries, and thus is strongly affected by the arteries' mechanical properties. This means the arteries along the pathway 151 associated with PTT2 may show less patient-to-patient variation in mechanical properties compared to arteries along the pathways 152, 153 associated with PTT1 and VTT. This may explain PTT2's relatively high correlation with blood pressure compared to PTT1 and VTT for the 110-patient study.

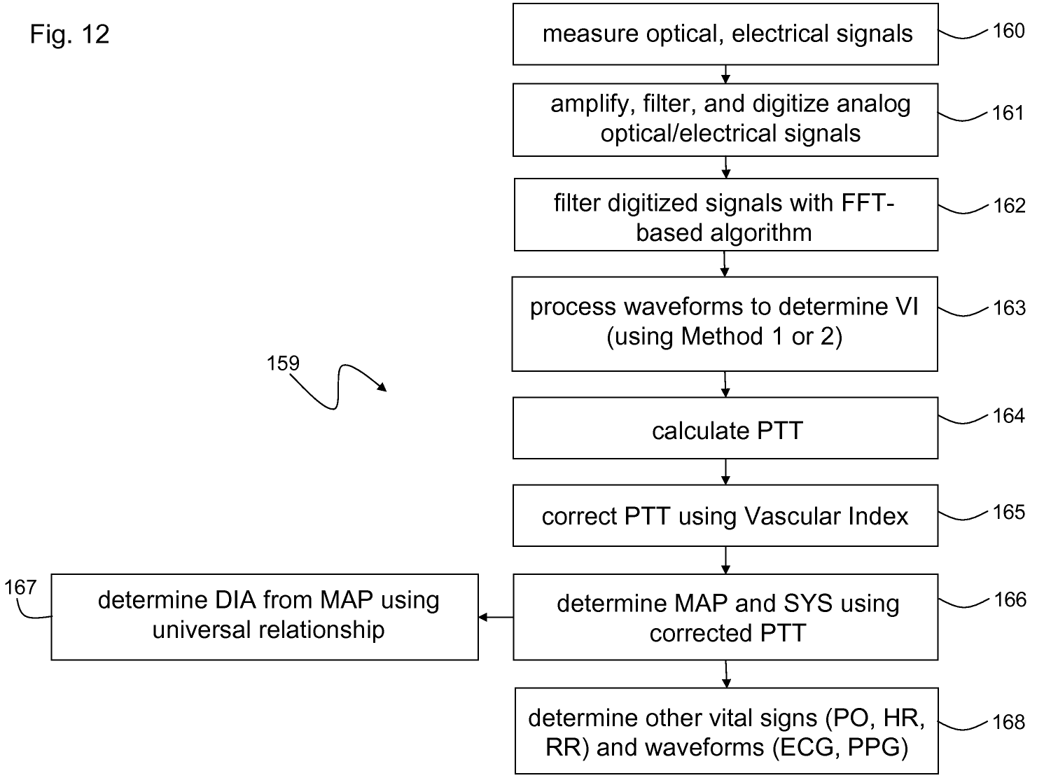

FIG. 12 shows a flowchart indicating an algorithm 59, based on the above-described study, which can be implemented with the device described above during a blood pressure measurement. Prior to the measurement, a caregiver (or in another implementation, the patient) attaches the sensor armband and sensors described in FIG. 1 to the patient. Once attached, the sensors simultaneously measure optical and electrical signals (step 160) as described above. These analog signals pass through into the electronics module on the sensor armband, where they are amplified (to increase signal strength) and filtered (to remove unwanted noise and correct for low-frequency modulation) with separate circuits, and finally digitized with an analog-to-digital converter (step 161). As shown in FIGS. 5A-C and 6, the digitized signals optical and electrical signals are then passed through a Windowed-Sinc Digital Filter to remove any unwanted noise (step 162). Once filtered, the resulting plethysmographs are processed by analyzing their second derivative as shown in FIGS. 4 and 7 and in Equations 1 and 2 to determine a VI for the patient according to Method 1 (step 163). Alternatively, VI can be estimated from VTT according to Method 2, as described in FIG. 8B and Equations 2 and 3. PTT (and most preferably PTT2) is measured from the optical and electrical waveforms as shown in FIGS. 8A and 10 (step 164), and then corrected for as described in Equation 2) using the VI (step 165). This correction accounts for patient-to-patient variation in arterial properties. Once corrected, PTT yields systolic and diastolic blood pressure using a predetermined mathematical relationship, e.g., a linear relationship characterized by a slope and y-intercept as described in Equations 4 and 5 (step 166). The slope and y-intercept of the mathematic relationship are determined prior to the measurement using a large (typically n>100) clinical study.

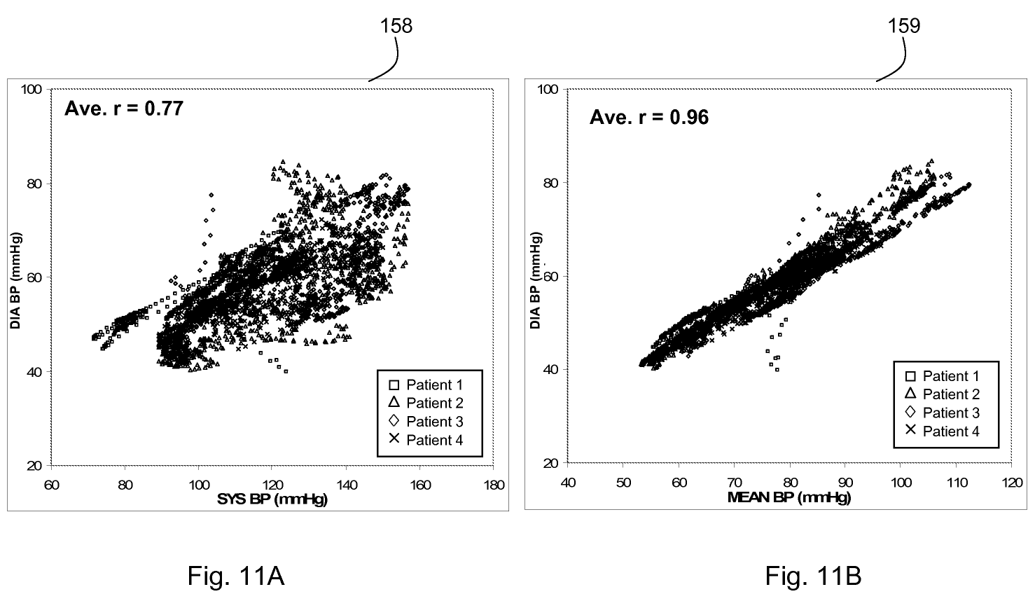

Diastolic blood pressure is determined from mean blood pressure using a universal relationship between these two parameters (step 167). For example, FIGS. 11A and 11B show mean, diastolic, and systolic blood pressure measured continuously from 4 patients during surgery using an arterial line. The figures show, respectively, linear relationships between diastolic blood pressure and systolic blood pressure (FIG. 11A) and diastolic blood pressure and mean blood pressure (FIG. 11B). They indicate that diastolic and mean blood pressure (r=0.96) correlate significantly better than diastolic and systolic blood pressure (r=0.77). This relationship has been verified with large numbers of patients using blood pressure values measured with both a pneumatic cuff and an arterial line. Following step 167, the algorithm yields systolic, diastolic, and mean arterial pressure.

Once blood pressure is determined, the optical and electrical waveforms can be further processed to determine other properties, such as heart rate, respiratory rate, and pulse oximetry (step 168). Pulse or heart rate, for example, is determined using techniques known in the art, e.g., determining the time spacing between pulses in the optical waveform, or QRS complexes in the electrical waveform, respectively. Respiratory rate modulates the time-dependent properties of the envelope of the optical and/or electrical waveforms, and thus can be determined, for example, by taking an FFT of these waveforms and analyzing low-frequency signals. Pulse oximetry can be determined from the optical waveform using well-known algorithms, such as those described in U.S. Pat. No. 4,653,498 to New, Jr. et al., the contents of which are incorporated herein by reference. Pulse oximetry requires time-dependent signals generated from two or more, separate and modulated light sources (in the red spectral range and in the infrared).

In addition to those methods described above, a number of additional methods can be used to calculate blood pressure from the optical and electrical waveforms. These are described in the following co-pending patent applications, the contents of which are incorporated herein by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL SIGN MONITOR FOR ATHLETIC APPLICATIONS (U.S. Ser. No; filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); and 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); 8) PATCH SENSOR FOR MEASURING BLOOD PRESSURE WITHOUT A CUFF (U.S. Ser. No. 10/906,315; filed Feb. 14, 2005); 9) PATCH SENSOR FOR MEASURING VITAL SIGNS (U.S. Ser. No. 11/160,957; filed Jul. 18, 2005); 10) WIRELESS, INTERNET-BASED SYSTEM FOR MEASURING VITAL SIGNS FROM A PLURALITY OF PATIENTS IN A HOSPITAL OR MEDICAL CLINIC (U.S. Ser. No. 11/162,719; filed Sep. 9, 2005); 11) HAND-HELD MONITOR FOR MEASURING VITAL SIGNS (U.S. Ser. No. 11/162,742; filed Sep. 21, 2005); 12) CHEST STRAP FOR MEASURING VITAL SIGNS (U.S. Ser. No. 11/306,243; filed Dec. 20, 2005); 13) SYSTEM FOR MEASURING VITAL SIGNS USING AN OPTICAL MODULE FEATURING A GREEN LIGHT SOURCE (U.S. Ser. No. 11/307,375; filed Feb. 3, 2006); 14) BILATERAL DEVICE, SYSTEM AND METHOD FOR MONITORING VITAL SIGNS (U.S. Ser. No. 11/420,281; filed May 25, 2006); and 15) SYSTEM FOR MEASURING VITAL SIGNS USING BILATERAL PULSE TRANSIT TIME (U.S. Ser. No. 11/420,652; filed May 26, 2006).

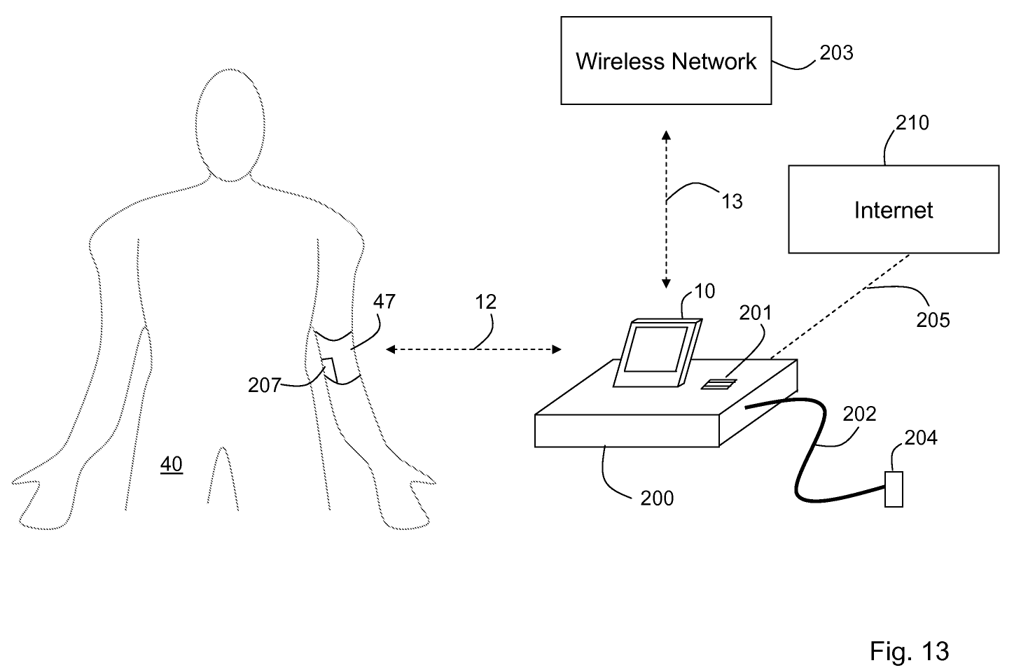

The above-described system can be used in a number of different settings, including both the home and hospital. FIG. 13, for example, shows a configuration suitable for both environments wherein a patient 40 continuously wears the sensor armband 47 over a time period ranging from minutes to several days. During this period, the sensor armband is powered by a rechargeable battery 207, and continuously measures blood pressure along with other vital signs. At a predetermined interval (typically, every few minutes) the sensor armband transmits this information through a short-range wireless interface 12 (e.g., a Bluetooth interface) to the device 10, which is seated in a docking station 200. The docking station 200 allows the device 10 to be easily seen by the patient or caregiver and additionally includes an AC adaptor 202 that plugs into a wall outlet 204 and continuously charges the device's battery as well as a spare battery 201 for the armband 47. When the original rechargeable battery 207 in the armband is depleted, the caregiver (or patient) 40 replaces it with the spare battery 201 in the docking station 200. The device 10 is highly portable and can be easily removed from the docking station 200. It communicates with a nation-wide wireless network 203 through a long-range wireless interface 13 (e.g., a CDMA modem), or with the Internet 210 through a wired interface 205.

Other embodiments are also within the scope of the invention. For example, software configurations other than those described above can be run on the device to give it a PDA-like functionality. These include, for example, Micro C OS®, Linux®, Microsoft Windows®, embOS, VxWorks, SymbianOS, QNX, OSE, BSD and its variants, FreeDOS, FreeRTOX, LynxOS, or eCOS and other embedded operating systems. The device can also run a software configuration that allows it to receive and send voice calls, text messages, or video streams received through the Internet or from the nation-wide wireless network it connects to. A bar-code scanner can also be incorporated into the device to capture patient or medical professional identification information, or other such labeling. This information, for example, can be used to communicate with a patient in a hospital or at home. In other embodiments, the device can connect to an Internet-accessible website to download content, e.g., calibrations, text messages, and information describing medications, from an associated website. As described above, the device can connect to the website using both wired (e.g., USB port) or wireless (e.g., short or long-range wireless transceivers) means. In still other embodiments, ‘alert’ values corresponding to vital signs and the pager or cell phone number of a caregiver can be programmed into the device using its graphical user interface. If a patient's vital signs meet an alert criteria, software on the device can send a wireless ‘page’ to the caregiver, thereby alerting them to the patient's condition.

Still other embodiments are within the scope of the following claims.

I claim as my invention:

1. A method for measuring a patient's blood pressure comprising:
measuring a first time-dependent optical signal with a first optical sensor;
measuring a second time-dependent optical signal with a second optical sensor;
measuring a time-dependent electrical signal with an electrical sensor;
estimating the patient's arterial properties from at least one of the first time-dependent optical signal or a derivative thereof, and the second time-dependent optical signal or a derivative thereof,
determining a pulse transit time from the time-dependent electrical signal or a derivative thereof, and at least one of the first and second time-dependent optical signals, or a derivative thereof, and
calculating a blood pressure using a mathematical model that includes the pulse transit time and the patient's arterial properties.

2. The method of claim 1, wherein determining the vascular transit time further comprises analyzing a first time-dependent feature from at least one of the first time-dependent optical signal or a derivative thereof, and a second time-dependent feature from the second time-dependent optical signal or a derivative thereof.

3. The method of claim 2, wherein the first time-dependent feature is comprised by a second derivative of an optical plethysmograph.

4. The method of claim 3, wherein the first time-dependent feature is a ratio of one or more peaks comprised a second derivative of an optical plethysmograph.

5. The method of claim 1, further comprising attaching the first optical sensor to a finger or wrist of the patient.

6. The method of claim 5, further comprising attaching the second optical sensor to a wrist or arm of the patient.

7. The method of claim 1, wherein the electrical sensor comprises at least two electrodes.

8. The method of claim 1, wherein a single sensor comprises at least one electrode and at least the first or second optical sensor.

9. The method of claim 1, wherein estimating the patient's arterial properties further comprises comparing a vascular transit time, or a derivative thereof, to a predetermined look-up table.

10. The method of claim 1, wherein estimating the patient's arterial properties further comprises comparing a vascular transit time, or a derivative thereof, to a mathematical function.

11. The method of claim 10, further comprising calculating a pulse wave velocity from the vascular transit time and a distance value corresponding to separation of the first and second optical sensors.

12. The method of claim 11, further comprising estimating the patient's arterial properties using the pulse wave velocity.

13. The method of claim 12, wherein estimating the patient's arterial properties further comprises comparing the pulse wave velocity, or a derivative thereof, to a predetermined look-up table.

14. The method of claim 12, wherein estimating the patient's arterial properties further comprises comparing the pulse wave velocity, or a derivative thereof, to a mathematical function.

15. The method of claim 1, wherein determining the pulse transit time further comprises analyzing a first time-dependent feature from the time-dependent electrical signal or a derivative thereof, and a second time-dependent feature from at least one of the first time-dependent optical signal or a derivative thereof, and a second time-dependent feature from the second time-dependent optical signal, or a derivative thereof.

16. The method of claim 15, wherein the first time-dependent feature comprises a peak corresponding to a portion of the time-dependent electrical signal.

17. The method of claim 15, wherein the second time-dependent feature comprises a base of an optical plethysmograph.

18. The method of claim 15, wherein the second time-dependent feature comprises a peak of an optical plethysmograph.

19. A device for measuring a patient's blood pressure, comprising:
a first optical sensor configured to measure a first time-dependent optical signal;
a second optical sensor configured to measure a second time-dependent optical signal;
an electrical sensor configured to measure a time-dependent electrical signal; and
a processor, in electrical communication with the first and second optical sensors and the electrical sensor; the processor configured to receive the first time-dependent optical signal or a derivative thereof, the second time-dependent optical signal or a derivative thereof, and the time-dependent electrical signal or a derivative thereof, the processor comprising a software program configured to:
i) estimate the patient's arterial properties from at least one of the first time-dependent optical signal or a derivative thereof, and the second time-dependent optical signal or a derivative thereof,
ii) determine a pulse transit time from the time-dependent electrical signal or a derivative thereof and either the first or second time-dependent optical signal or a derivative thereof, and
iii) calculate a blood pressure value using a mathematical model that includes the pulse transit time and the patient's arterial properties.

20. A device for measuring a patient's blood pressure, comprising:
a first optical sensor configured to measure a first time-dependent optical signal;
a second optical sensor configured to measure a second time-dependent optical signal;
an electrical sensor configured to measure a time-dependent electrical signal; and
a processor configured to:
i) process the first time-dependent optical signal or a derivative thereof, to generate a first processed optical signal;
ii) process the second time-dependent optical signal or a derivative thereof, to generate a second processed optical signal;
iii) process the time-dependent electrical signal or a derivative thereof, to generate a processed electrical signal;
iv) estimate arterial properties from at least one of the first processed optical signal and the second processed optical signal;
v) determine a pulse transit time from the processed electrical signal and at least one of the first processed optical signal and the second processed optical signal; and,
iv) calculate a blood pressure value using the pulse transit time and the estimated arterial properties.

21. A method for measuring a patient's blood pressure comprising:
measuring a first time-dependent optical signal with a first optical sensor disposed on the patient's finger;
measuring a second time-dependent optical signal with a second optical sensor disposed on the patient's arm;
measuring a time-dependent electrical signal with an electrical sensor comprising at least two electrodes;
determining a pulse wave velocity from the first time-dependent optical signal or a derivative thereof, the second time-dependent optical signal or a derivative thereof, and a distance separating the first optical sensor and the second optical sensor;
estimating the patient's arterial properties using the pulse wave velocity, or a derivative thereof;
determining a pulse transit time from the time-dependent electrical signal or a derivative thereof and at least one of the first and second time-dependent optical signal, or a derivative thereof; and,
calculating a blood pressure value using a mathematical model that includes the pulse transit time and the patient's arterial properties.
